(19) United States
(12) Patent Application Publication
(10) Pub. No.: US 2007/0142435 A1
(43) Pub. Date: Jun. 21, 2007

(54) [4-(5-Aminomethyl-2-fluoro-phenyl)-piperidin-1-yl]-(4-bromo-3-methyl-5-propoxy-thiophen-2-yl)-methanone hydrochloride as an inhibitor of mast cell tryptase

(75) Inventors: Zhongli Gao, Flemington, NJ (US); Larry Davis, Sergeantsville, NJ (US); Julian Levell, Arlington, MA (US); Mark Czekaj, Doylestown, NJ (US); Adam W. Sledeski, Belle Mead, NJ (US)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater, NJ (US)

(21) Appl. No.: 11/520,998

(22) Filed: Sep. 14, 2006

Related U.S. Application Data
(63) Continuation of application No. PCT/US05/09899, filed on Mar. 24, 2005.
(60) Provisional application No. 60/556,927, filed on Mar. 26, 2004.

(51) Int. Cl. A61K 31/453; C07D 409/02

(52) U.S. Cl. 514/326; 546/212

(57) ABSTRACT

The present invention extends to the compound of formula I: 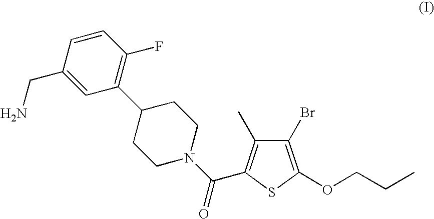 or a prodrug, pharmaceutically acceptable salt, or solvate of said compound. Furthermore, the present invention is directed to a pharmaceutical composition comprising a pharmaceutically effective amount of the compound of formula I, and a pharmaceutically acceptable carrier.

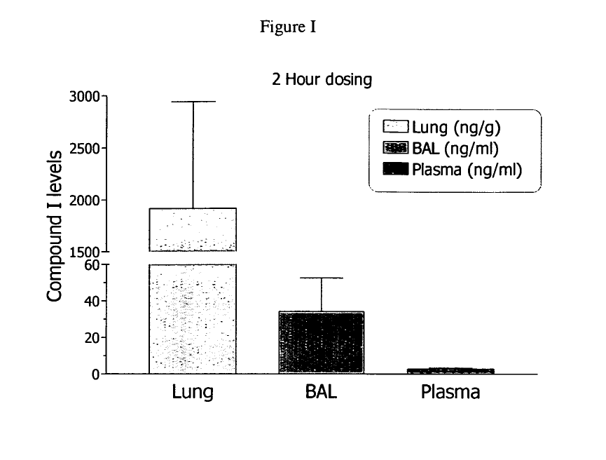

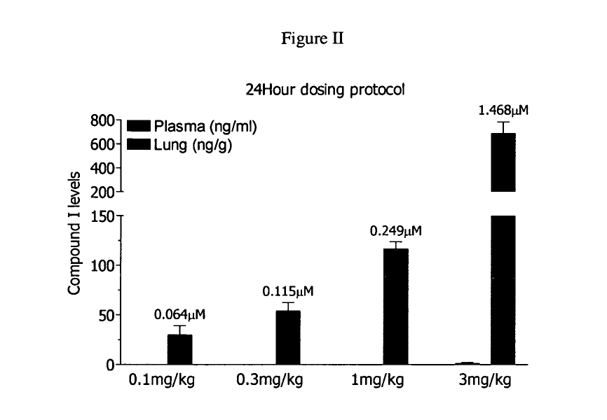

Furthermore, the present invention is directed to the use of a compound of formula I as an inhibitor of tryptase, comprising introducing the compound into a composition comprising tryptase. In addition, the present invention is directed to the use of a compound of formula I for treating a patient suffering from, or subject to, a physiological condition in need of amelioration of an inhibitor of tryptase comprising administering to the patient a therapeutically effective amount of the compound of claim 1 The present invention is directed also to the preparation of a compound of formula I.

### CROSS-REFERENCE TO RELATED APPLICATIONS

This application is a continuation of WO application No. PCT/US/2005/009899, filed Mar. 24, 2005, which claims the benefit of priority from U.S. provisional application No. 60/556,927, filed on Mar. 26, 2004.

### FIELD OF THE INVENTION

This invention is directed to a substituted arylmethylamine compound, its preparation, a pharmaceutical composition comprising the compound, its use, and intermediates thereof.

### BACKGROUND OF THE INVENTION

Mast cell mediated inflammatory conditions, in particular asthma, are a growing public health concern. Asthma is frequently characterized by progressive development of hyper-responsiveness of the trachea and bronchi to both immunospecific allergens and generalized chemical or physical stimuli, which lead to the onset of chronic inflammation. Leukocytes containing IgE receptors, notably mast cells and basophils, are present in the epithelium and underlying smooth muscle tissues of bronchi. These leukocytes initially become activated by the binding of specific inhaled antigens to the IgE receptors and then release a number of chemical mediators. For example, degranulation of mast cells leads to the release of proteoglycans, peroxidase, arylsulfatase B, chymase, and tryptase, which results in bronchiole constriction.

Tryptase is stored in the mast cell secretory granules and is the major secretory protease of human mast cells. Tryptase has been implicated in a variety of biological processes, including degradation of vasodilating and bronchorelaxing neuropeptides (Caughey, et al., J. Pharmacol. Exp. Ther., 1988, 244, pages 133-137; Franconi, et al., J. Pharmacol. Exp. Ther., 1988, 248, pages 947-951; and Tam, et al., Am. J. Respir. Cell Mol. Biol., 1990, 3, pages 27-32) and modulation of bronchial responsiveness to histamine (Sekizawa, et al., J. Clin. Invest., 1989, 83, pages 175-179).

As a result, tryptase inhibitors may be useful as anti-inflammatory agents (K Rice, P. A. Sprengler, Current Opinion in Drug Discovery and Development, 1999, 2(5), pages 463474) particularly in the treatment of chronic asthma (M. Q. Zhang, H. Timmerman, Mediators Inflamm., 1997, 112, pages 311-317), and may also be useful in treating or preventing allergic rhinitis (S. J. Wilson et al, Clin. Exp. Allergy, 1998, 28, pages 220-227), inflammatory bowel disease (S. C. Bischoff et al, Histopathology, 1996, 28, pages 1-13), psoriasis (A. Naukkarinen et al, Arch. Dermatol. Res., 1993, 285, pages 341-346), conjunctivitis (A. A. Irani et al, J. Allergy Clin. Immunol., 1990, 86, pages 3440), atopic dermatitis (A. Jarvikallio et al, Br. J. Dermatol., 1997, 136, pages 871-877), rheumatoid arthritis (L. C. Tetlow et al, Ann. Rheum. Dis., 1998, 54, pages 549-555), osteoarthritis (M. G. Buckley et al, J. Pathol., 1998, 186, pages 67-74), gouty arthritis, rheumatoid spondylitis, and diseases of joint cartilage destruction.

In addition, tryptase has been shown to be a potent mitogen for fibroblasts, suggesting its involvement in the pulmonary fibrosis in asthma and interstitial lung diseases (Ruoss et al., J. Clin. Invest., 1991, 88, pages 493499).

Therefore, tryptase inhibitors may be useful in treating or preventing fibrotic conditions (J. A. Cairns and A. F. Walls, J. Clin. Invest., 1997, 99, pages 1313-1321) for example, fibrosis, sceleroderma, pulmonary fibrosis, liver cirrhosis, myocardial fibrosis, neurofibromas and hypertrophic scars.

Additionally, tryptase inhibitors may be useful in treating or preventing myocardial infarction, stroke, angina and other consequences of atherosclerotic plaque rupture (M. Jeziorska et al, J. Pathol., 1997, 182, pages 115-122).

Tryptase has also been discovered to activate prostromelysin that in turn activates collagenase, thereby initiating the destruction of cartilage and periodontal connective tissue, respectively.

Therefore, tryptase inhibitors could be useful in the treatment or prevention of arthritis, periodontal disease, diabetic retinopathy, and tumor growth (W. J. Beil et al, Exp. Hematol., (1998) 26, pages 158-169). Also, tryptase inhibitors may be useful in the treatment of anaphylaxis (L. B. Schwarz et al, J. Clin. Invest., 1995, 96, pages 2702-2710), multiple sclerosis (M. Steinhoff et al, Nat. Med. (N. Y.), 2000, 6(2), pages 151-158), peptic ulcers and syncytial viral infections.

Substituted arylmethylamines, represented as the compounds of formula (A), their preparation, pharmaceutical compositions containing these compounds, and their pharmaceutical use in the treatment of disease states capable of being modulated by the inhibition of tryptase are reported in pending U.S. application Ser. No. 09/843,126. Encompassed within the generic disclosure of the compounds of formula (A) of U.S. application Ser. No. 09/843,126, is the compound of the present invention, formula I. However, the compound of formula I is not specifically disclosed in U.S. application Ser. No. 09/843,126.

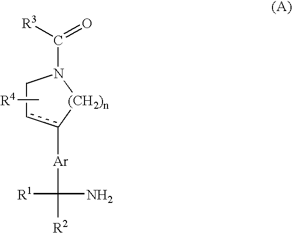

Accordingly, what is needed is a novel and useful compound having particularly valuable pharmaceutical properties, in its ability to inhibit tryptase. Such a compound should readily have a utility in treating a patient suffering from conditions that can be ameliorated by the administration of an inhibitor of tryptase, e.g., mast cell mediated inflammatory conditions, inflammation, and diseases or disorders related to the degradation of vasodilating and bronchorelaxing neuropeptides.

### SUMMARY OF THE INVENTION

The present invention extends to the compound of formula I: 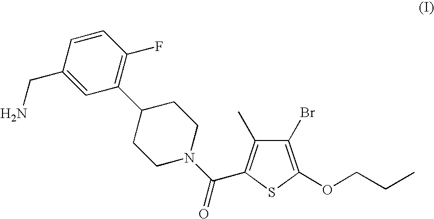
or a prodrug, pharmaceutically acceptable salt, or solvate of said compound.

Furthermore, the present invention is directed to a pharmaceutical composition comprising a pharmaceutically effective amount of the compound of formula I, and a pharmaceutically acceptable carrier.

Furthermore, the present invention is directed to the use of a compound of formula I as an inhibitor of tryptase, comprising introducing the compound into a composition comprising tryptase. In addition, the present invention is directed to the use of a compound of formula I for treating a patient suffering from, or subject to, a physiological condition in need of amelioration of an inhibitor of tryptase comprising administering to the patient a therapeutically effective amount of the compound of claim 1.

The present invention is directed also to the preparation of a compound of formula I.

### BRIEF DESCRIPTION OF THE DRAWINGS

Aspects, features and advantages of the present invention will be better understood from the following detailed descriptions taken in conjunction with the accompanying Figures, all of which are given by way of illustration only, and are not limitative of the present invention, in which:

FIG. I: Compound I levels in plasma, bronchoalveolar lavage (BAL) fluid and lung, measured 2 hour after dosing compound 1 at 1 mg/kg, p.o. Values are mean±SE of 6-8 animals.

FIG. II: Plasma and lung compound I levels measured 24 hour post dosing. Values are mean±SE of 3-4 animals.

### DETAILED DESCRIPTION

Definitions

As used above, and throughout the instant specification and appending claims, the following terms, unless otherwise indicated, shall be understood to have the following meanings:

As used herein, the term “compound of the present invention”, and equivalent expressions, are meant to embrace the compound of formula I, as hereinbefore described, which expression includes the prodrug, the pharmaceutically acceptable salt and the solvate, e.g., hydrate. Similarly, reference to intermediates, whether or not they themselves are claimed, is meant to embrace the salts, and solvates, where the context so permits. For the sake of clarity, particular instances when the context so permits are sometimes indicated in the text, but these instances are purely illustrative and they are not intended to exclude other instances when the context so permits.

As used herein, the term “treatment” or “treating” includes prophylactic therapy as well as treatment of an established condition.

“Patient” means a human or other mammal.

“Effective amount” is meant to describe an amount of a compound effective in producing the desired therapeutic effect.

“Prodrug” means a compound that is suitable for administration to a patient without undue toxicity, irritation, allergic response, and the like, and is convertible in vivo by metabolic means (e.g. by hydrolysis) to the compound of the present invention. A thorough discussion of prodrugs is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987, both of which are incorporated herein by reference.

Particular or Preferred Embodiments

In addition, the present invention is directed to the use of the compound of formula I for treating a patient suffering from a physiological condition that can be ameliorated by administering to the patient a therapeutically effective amount of the compound of formula I. Particular embodiments of physiological conditions that can be treated with the compound of the present invention include, but certainly are not limited to inflammatory diseases, e.g., joint inflammation, arthritis, rheumatoid arthritis, rheumatoid spondylitis, gouty arthritis, traumatic arthritis, rubella arthritis, psoriatic arthritis, and other chronic inflammatory joint diseases. Other embodiments of physiological conditions that can be treated by the present invention include physiological conditions such as chronic obstructive pulmonary disease (COPD), COPD exacerbations, joint cartilage destruction,ocular conjunctivitis, vernal conjunctivitis, inflammatory bowel disease, asthma, allergic rhinitis, interstitial lung diseases, fibrosis, sceleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina and other consequences of atherosclerotic plaque rupture, as well as periodontal disease, diabetic retinopathy, tumor growth, anaphylaxis, multiple sclerosis, peptic ulcers, and syncytial viral infections.

In a particular embodiment, the present invention is directed to the use of a compound of formula I for treating a patient suffering from asthma, comprising administering to the patient a physiologically effective amount of the compound.

In another particular embodiment, the present invention is directed to the use of a compound of formula I for treating a patient suffering from COPD, comprising administering to the patient a physiologically effective amount of the compound.

In another particular embodiment, the present invention is directed to the use of a compound of formula I for treating a patient suffering from COPD exacerbations, comprising administering to the patient a physiologically effective amount of the compound.

In another particular embodiment, the present invention is directed to the use of a compound of formula I for treating a patient suffering from allergic rhinitis, comprising administering to the patient a physiologically effective amount of the compound.

In another particular embodiment, the present invention is directed to the use of a compound of formula I for treating a patient suffering from joint inflammation, comprising administering to the patient a physiologically effective amount of the compound.

In another particular embodiment, the present invention is directed to the use of a compound of formula I for treating a patient suffering from inflammatory bowel disease, comprising administering to the patient a physiologically effective amount of the compound.

In addition, the present invention extends to a pharmaceutical composition comprising the compound of formula I, a second compound selected from the group consisting of a beta andrenergic agonist, an anticholinergic, an anti-inflammatory corticosteroid, and an anti-inflammatory agent, and a pharmaceutically acceptable carrier thereof. In such a composition the compound of formula I and the second compound are present in amounts such that provide a therapeutically efficacious activity, i.e., additive or synergistic effect. Particular inflammatory diseases or disorders that can be treated with such a pharmaceutical composition include, but is not limited to, asthma.

Moreover, the present invention is directed to a method for treating a patient suffering from an inflammatory disorder, comprising administering to the patient the compound of formula I and a second compound selected from the group consisting of a beta andrenergic agonist, an anticholinergic, an anti-inflammatory corticosteroid, and an anti-inflammatory agent. In such a method, the compound of formula I and the second compound are present in amounts such that provide a therapeutically efficacious activity, i.e., additive or synergistic effect. In such a method of the present invention, the compound of the present invention can be administered to the patient before a second compound, a second compound can be administered to the patient before a compound of the present invention, or a compound of the present invention and a second compound can be administered concurrently. Particular examples of andrenergic agonists, anticholinergics, anti-inflammatory corticosteroids, and anti-inflammatory agents having application according to the method are described infra.

Pharmaceutical Compositions

As explained above, the compound of the present invention exhibits useful pharmacological activity and accordingly may be incorporated into a pharmaceutical composition and used in the treatment of patients suffering from certain medical disorders. The present invention thus provides, according to a further aspect, pharmaceutical compositions comprising the compound of the invention, and a pharmaceutically acceptable carrier thereof. As used herein, the term “pharmaceutically acceptable” preferably means approved by a regulatory agency of a government, in particular the Federal government or a state government, or listed in the U.S. Pharmacopoeia or another generally recognized pharmacopoeia for use in animals, and more particularly in humans. Suitable pharmaceutical carriers are described in “Remington's Pharmaceutical Sciences” by E. W. Martin.

Pharmaceutical compositions according to the present invention can be prepared according to the customary methods, using one or more pharmaceutically acceptable adjuvants or excipients. The adjuvants comprise, inter alia, diluents, fillers, binders, disintegrants, glidants, lubricants, surfactants, sterile aqueous media and the various non-toxic organic solvents. The compositions may be presented in the form of tablets, capsules, pills, sustained release formulations, granules, powders, aqueous solutions or suspensions, injectable solutions, elixirs or syrups, and can contain one or more agents chosen from the group comprising sweeteners, flavorings, colorings, or stabilizers in order to obtain pharmaceutically acceptable preparations. The choice of vehicle and the content of active substance in the vehicle are generally determined in accordance with the solubility and chemical properties of the active compound, the particular mode of administration and the provisions to be observed in pharmaceutical practice. For example, excipients such as lactose, microcrystalline cellulose, pregelatinized starch, unmodified starch, silicified microcrystalline cellulose, mannitol, sorbitol, xylitol, dextrates, fructose, sodium citrate, calcium carbonate, dicalcium phosphate dihydrate, anhydrous dicalcium phosphate, calcium sulfate, along with binders such as polyvinylpyrollidone, hydroxypropylmethyl cellulose, ethyl cellulose, hydroxyethyl cellulose, methyl cellulose, sodium carboxymethyl cellulose, pregelatinized starch, starch, polyethylene glycols, polyethylene oxide, polycarbophils, gelatin and acacia and disintegrating agents such as sodium croscarmellose, sodium starch glycolate, crospovidone, starch, microcrystalline cellulose, alginic acids and certain complex silicates combined with lubricants such as magnesium stearate, calcium stearate, stearic acid, hydrogenated vegetable oil, mineral oil, polyethylene glycols, glyceryl esters of fatty acids, sodium lauryl sulfate and glidants such as silicon dioxide, talc, starch, along with some suitable wetting agent such as sodium lauryl sulfate, sorbitan esters, polyoxyethylene fatty acid esters, poloxamer, polyoxyethylene ether, sodium docusate, polyethoxylated castor oil, and benzalkonium chloride may be used for preparing tablets. To prepare a capsule, it is advantageous to use fillers such as lactose, microcrystalline cellulose, pregelatinized starch, unmodified starch, silicified microcrystalline cellulose alone or a mixture of two or more fillers, with and without binders as described above along with suitable wetting agent (s), disintegrants, glidants, lubricants, etc. as listed above. When aqueous suspensions are used they can contain emulsifying agents or agents which facilitate suspension. Diluents such as sucrose, ethanol, polyethylene glycol, propylene glycol, glycerol and chloroform or mixtures thereof may also be used. Such pharmaceutically acceptable carriers can also be sterile water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include mannitol, human serum albumin (HSA), starch, glucose, lactose, sucrose, gelatin, malt, rice, flour, chalk, silica gel, magnesium carbonate, magnesium stearate, sodium stearate, glycerol monostearate, talc, sodium chloride, dried skim milk, glycerol, propylene, glycol, water, ethanol and the like. These compositions can take the form of solutions, suspensions, tablets, pills, capsules, powders, sustained-release formulations and the like.

Naturally, a pharmaceutical composition of the present invention will contain a therapeutically effective amount of the active compound together with a suitable amount of carrier so as to provide the form for proper administration to the patient. While intravenous injection is a very effective form of administration, other modes can be employed, such as by injection, or by oral, nasal or parenteral administration, which are discussed infra.

Methods of Treatment

The compound of formula I possesses tryptase inhibition activity according to tests described in the literature and described hereinafter, and which test results are believed to correlate to, pharmacological activity in humans and other mammals. Thus, in a further embodiment, the present invention is directed to the use of formula I or a composition comprising it for treating a patient suffering from, or subject to, a condition that can be ameliorated by the administration of an inhibitor of tryptase. For example, the compound of formula I is useful for treating an inflammatory disease, for example, joint inflammation, including arthritis, rheumatoid arthritis and other arthritic condition such as rheumatoid spondylitis, gouty arthritis, traumatic arthritis, rubella arthritis, psoriatic arthritis, osteoarthritis or other chronic inflammatory joint disease, or diseases of joint cartilage destruction, Cular conjunctivitis, vernal conjunctivitis, inflammatory bowel disease, asthma, allergic rhinitis, interstitial lung diseases, fibrosis, sceleroderma, pulmonary fibrosis, liver cirrhosis, myocardial fibrosis, neurofibromas, hypertrophic scars, various dermatological conditions, for example, atopic dermatitis and psoriasis, myocardial infarction, stroke, angina or other consequences of atherosclerotic plaque rupture, as well as periodontal disease, diabetic retinopathy, tumor growth, anaphylaxis, multiple sclerosis, peptic ulcers, or a syncytial viral infection.

According to a further feature of the invention there is provided a method for the treatment of a human or animal patient suffering from, or subject to, conditions which can be ameliorated by the administration of an inhibitor of tryptase, for example conditions as hereinbefore described, which comprises the administration to the patient of an effective amount of compound of the invention or a composition containing a compound of the invention.

Combination Therapy

As explained above, other pharmaceutically active agents can be employed in combination with the compound of formula I depending upon the disease being treated. For example, in the treatment of asthma, beta-adrenergic agonists such as albuterol, terbutaline, formoterol, fenoterol or prenaline can be included, as can anticholinergics such as ipratropium bromide, anti-inflammatory corticosteroids such as beclomethasone dipropionate, triamcinolone acetonide, flunisolide or dexamethasone, and anti-inflammatory agents such as sodium cromoglycate and nedocromil sodium. Thus, the present invention extends to a pharmaceutical composition comprising the compound of formula I and a second compound selected from the group consisting of a beta andrenergic agonist, an anticholinergic, an anti-inflammatory corticosteroid, and an anti-inflammatory agent; and a pharmaceutically acceptable carrier thereof. Particular pharmaceutical carriers having applications in this pharmaceutical composition are described herein.

Furthermore, the present invention extends to a method for treating a patient suffering from asthma, comprising administering the patient the compound of the present invention, and a second compound selected from the group consisting of a beta andrenergic agonist, an anticholinergic, an anti-inflammatory corticosteroid, and an anti-inflammatory agent. In such a combination method, the compound of the present invention can be administered prior to the administration of the second compound, the compound of the present invention can be administered after administration of the second compound, or the compound of the present invention and the second compound can be administered concurrently.

Modes of Delivery

According to the invention, the compound of formula I, or a pharmaceutical composition comprising the compound, may be introduced parenterally, transmucosally, e.g., orally, nasally, pulmonarily, or rectally, or transdermally to a patient.

Oral Delivery

Contemplated for use herein are oral solid dosage forms, which are described generally in Remington's Pharmaceutical Sciences, 18th Ed.1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for a therapeutic is given by Marshall, K. In: Modern Pharmaceutics Edited by G. S. Banker and C. T. Rhodes Chapter 10, 1979, herein incorporated by reference. In general, the formulation will include a compound of the present invention, and inert ingredients that allow for protection against the stomach environment, and release of the biologically active material, i.e., a compound of the present invention, in the intestine.

Also specifically contemplated are oral dosage forms of the compound of the present invention. Such a compound may be chemically modified so that oral delivery is more efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the compound of the present invention, and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the compound of the present invention, the location of release may be the stomach, the small intestine (the duodenum, the jejunum, or the ileum), or the large intestine. One skilled in the art has available formulations that will not dissolve in the stomach, yet will release the material in the duodenum or elsewhere in the intestine. Preferably, the release will avoid the deleterious effects of the stomach environment, either by protection of the compound of the present invention, or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings that make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the present invention may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include, but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of a compound of the present invention either alone or as a mixture in different ratios.

Additives that potentially enhance uptake of the compound of the present invention are, for instance, the fatty acids oleic acid, linoleic acid and linolenic acid. Controlled release oral formulation may be desirable. The drug could be incorporated into an inert matrix that permits release by either diffusion or leaching mechanisms, e.g., gums. Slowly degenerating matrices may also be incorporated into the formulation. Some enteric coatings also have a delayed release effect. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Other coatings may be used for the formulation. These include a variety of sugars that could be applied in a coating pan. The therapeutic agent could also be given in a film-coated tablet and the materials used in this instance are divided into 2 groups. The first are the non-enteric materials and include methyl cellulose, ethyl cellulose, hydroxyethyl cellulose, methylhydroxyethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan-coater or in a fluidized bed or by compression coating.

Pulmonarv Delivery

Also contemplated herein is pulmonary delivery of the compound of the present invention, either alone, or in a pharmaceutical composition. The compound is delivered to the lungs of a mammal while inhaling and traverses across the lung epithelial lining to the blood stream. Other reports of this include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (al-antitrypsin); Smith et al., 1989, J.Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-y and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass., to name only a few. All such devices require the use of formulations suitable for the dispensing of the compound of the present invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. A chemically modified compound of the present invention may also be prepared in different formulations depending on the type of chemical modification or the type of device employed.

Formulations suitable for use with a nebulizer, either jet or ultrasonic, will typically comprise the compound of the present invention dissolved in water at a concentration of about 0.1 to 25 mg of compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the compound of the invention suspended in a propellant with the aid of a surfactant. The propellant may be any conventional material employed for this purpose, such as a chlorofluorocarbon, hydrochlorofluorocarbon, hydrofluorocarbon, or hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the compound of the invention, and may also include a bulking agent, such as lactose, sorbitol, sucrose, or mannitol in amounts which facilitate dispersal of the powder from the device, e.g., 50 to 90% by weight of the formulation. The compound of the present invention should most advantageously be prepared in particulate form with an average particle size of less than 10 mm (or microns), most preferably 0.5 to 5 mm, for most effective delivery to the distal lung.

Nasal Delivery

Nasal delivery of the compound of the present invention is also contemplated. Nasal delivery allows the passage of the compound to the blood stream directly after administering the therapeutic product to the nose, without the necessity for deposition of the product in the lung. Formulations for nasal delivery include those with dextran or cyclodextran.

Transdermal Delivery

Various and numerous methods are known in the art for transdermal administration of a drug, e.g., via a transdermal patch, have applications in the present invention. Transdermal patches are described in for example, U.S. Pat. No. 5,407,713, issued Apr. 18, 1995 to Rolando et al.; U.S. Pat. No. 5,352,456, issued Oct. 4, 1994 to Fallon et al.; U.S. Pat. No. 5,332,213 issued Aug. 9, 1994 to D'Angelo et al.; U.S. Pat. No. 5,336,168, issued Aug. 9, 1994 to Sibalis; U.S. Pat. No. 5,290,561, issued Mar. 1, 1994 to Farhadieh et al.; U.S. Pat. No. 5,254,346, issued Oct. 19, 1993 to Tucker et al.; U.S. Pat. No. 5,164,189, issued Nov. 17, 1992 to Berger et al.; U.S. Pat. No. 5,163,899, issued Nov. 17, 1992 to Sibalis; U.S. Pat. Nos. 5,088,977 and 5,087,240, both issued Feb. 18, 1992 to Sibalis; U.S. Pat. No. 5,008,110, issued Apr. 16, 1991 to Benecke et al.; and U.S. Pat. No. 4,921,475, issued May 1, 1990 to Sibalis, the disclosure of each of which is incorporated herein by reference in its entirety.

It can be readily appreciated that a transdermal route of administration may be enhanced by use of a dermal penetration enhancer, e.g., such as enhancers described in U.S. Pat. No. 5,164,189 (supra), U.S. Pat. No. 5,008,110 (supra), and U.S. Pat. No. 4,879,119, issued Nov. 7, 1989 to Aruga et al., the disclosure of each of which is incorporated herein by reference in its entirety.

Topical Administration

For topical administration, gels (water or alcohol based), creams or ointments containing compounds of the invention may be used. Compounds of the invention may also be incorporated in a gel or matrix base for application in a patch, which would allow a controlled release of compound through the transdermal barrier.

Rectal Administration

Solid compositions for rectal administration include suppositories formulated in accordance with known methods and containing the compound of the invention.

Dosages

The percentage of active ingredient in the composition of the invention may be varied, it being necessary that it should constitute a proportion such that a suitable dosage shall be obtained. Obviously, several unit dosage forms may be administered at about the same time. The dose employed will be determined by the physician, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 1, mg/kg body weight per day by intravenous administration. In each particular case, the doses will be determined in accordance with the factors distinctive to the subject to be treated, such as age, weight, general state of health and other characteristics which can influence the efficacy of the medicinal product.

Furthermore, the compound according to the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. Of course, for some patients, it will be necessary to prescribe not more than one or two doses per day.

Naturally, a patient in whom administration of the compound of the present invention is an effective therapeutic regimen is preferably a human, but can be any animal. Thus, as can be readily appreciated by one of ordinary skill in the art, the methods and pharmaceutical compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

Preparatory Details

The compound of formula I may be prepared by the application or adaptation of known methods, by which is meant methods used heretofore or described in the literature, for example those described by R. C. Larock in Comprehensive Organic Transformations, VCH publishers, 1989, or as described herein.

In the reactions described hereinafter it may be necessary to protect reactive functional groups, for example, amino groups, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991. In particular, the compound of formula I may be prepared as shown through Schemes I-III.

For example, the compound of the present invention is an achiral compound whose preparation is comprised of a convergent synthesis. Scheme I below shows the procedures that culminate in the preparation of amine 10. Scheme II below shows the procedures that culminate in the preparation of acid 16. Scheme III below shows the procedures that culminate in the preparation of the compound of formula I, to yield the compound of formula I in a two-step sequence. The preparation of 10, 16 and the compound of formula I of the present invention are discussed in turn below.

Compound 2 is converted to compound 3 by protecting the amino group with an amino protecting agent, such as 1,2-bis(chlorodimethylsilyl)ethane, in the presence of a tertiary amine, such as triethylamine, in a suitable inert solvent, such as dichloromethane, to yield protected compound 3.

Compound 3 is converted to compound 5 by alkylating compound 4 using compound 3 under alkylating conditions comprising a strong base, such as n-butyllithium, in a suitable aprotic solvent, such as tetrahydrofuran, to yield the hydroxyl derivative compound 5.

Compound 5 is converted to compound 6 by deprotecting the amino group thereof with a deprotecting agent, such as a strong inorganic acid, such as phosphoric acid, in the presence of an inert solvent, such as heptane, to yield deprotected compound 6.

Compound 6 is converted to compound 7 by dehydrating, using an strong inorganic acid, such as phosphoric acid, and subsequent neutralizing the product using a strong inorganic base, such as aqueous sodium hydroxide to yield the dehydrated compound 7.

Compound 7 is converted to compound 8 by protecting the amino group with an amino protecting agent, such as boc anhydride, in a mixed aqueous/organic solvent system, wherein the organic solvent is a polar organic solvent such as methanol, using a strong inorganic base, such as aqueous sodium hydroxide to yield boc-protected compound 8.

Compound 8 is converted to compound 9 by hydrogenating using a reducing agent, such as palladium hydroxide on carbon (20%), in a mixed solvent system, such as methanolic acetic acid to yield the deprotected piperidine salt, compound 9.

Compound 9 is converted to its free base form, compound 10, by neutralizing using a strong inorganic base, such as aqueous sodium hydroxide, to yield final compound 10.

Carbon disulfide is converted to compound 11 by acylating using the acylating agent propyl chloroformate in the presence of a strong inorganic base, such as potassium hydroxide, in n-propanol to yield compound 11.

Compound 11 is converted to compound 12 by alkylating acetone with compound 11 in the presence of a strong base such as sodium hydride to yield alkylated compound 12.

Compound 12 is converted to compound 13 by alkylating compound 12 with α-bromo methyl acetate in the presence of a tertiary amine such as triethylamine to yield the alkylated compound 13.

Compound 13 is converted to compound 14 by cyclizing under strong base conditions, such as sodium methoxide, in the presence of a protic, organic solvent such as methanol to yield the cyclized compound 14.

Compound 14 is converted to compound 15 by brominating in an inert organic solvent, such as trichloromethane or a TBME/heptane mixture, to yield the brominated compound 15.

Compound 15 is converted to compound 16 by hydrolyzing using a strong inorganic base, such as lithium hydroxide.

Compound 16 is converted to compound 17 by coupling with compound 10 using a coupling agent, such as TPTU/HOBT or EDC, in the presence of an inert solvent, such as dichloromethane, and a tertiary amine such as diisopropyl ethylamine under anhydrous conditions, to yield the coupled compound 17.

Compound 17 is converted to compound I by deprotecting under strong acid conditions, such as hydrochloric acid, in the presence of a polar organic solvent, such as dioxane, to yield the deprotected compound I.

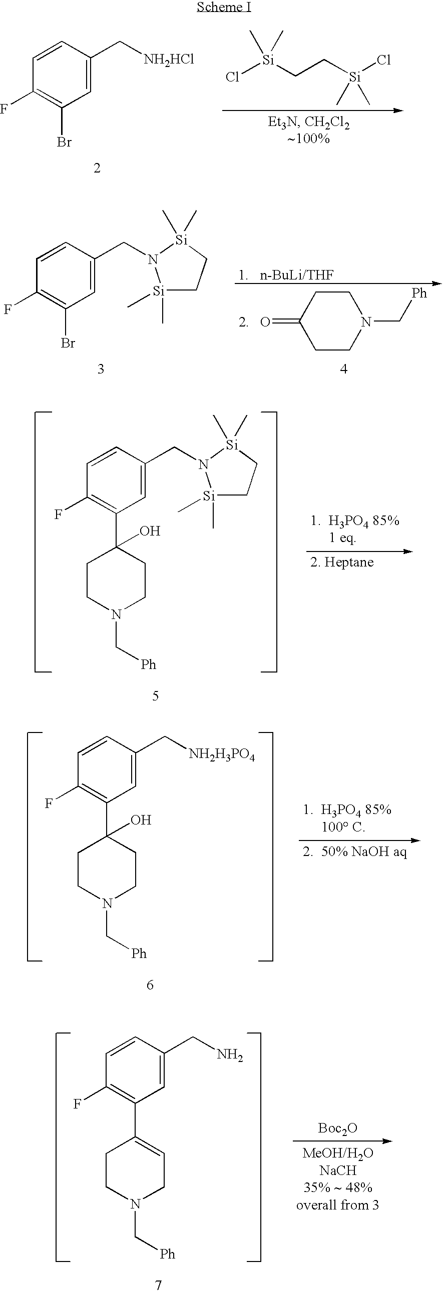

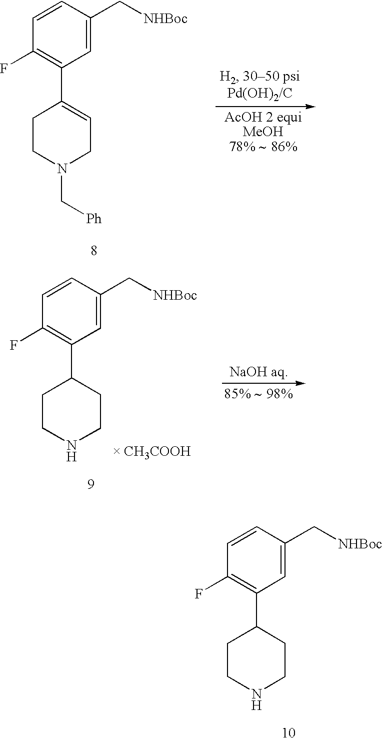

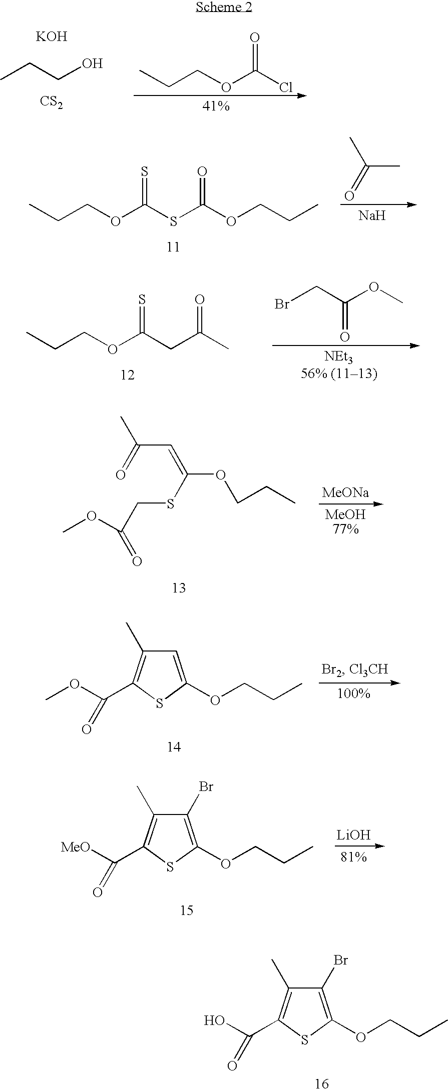

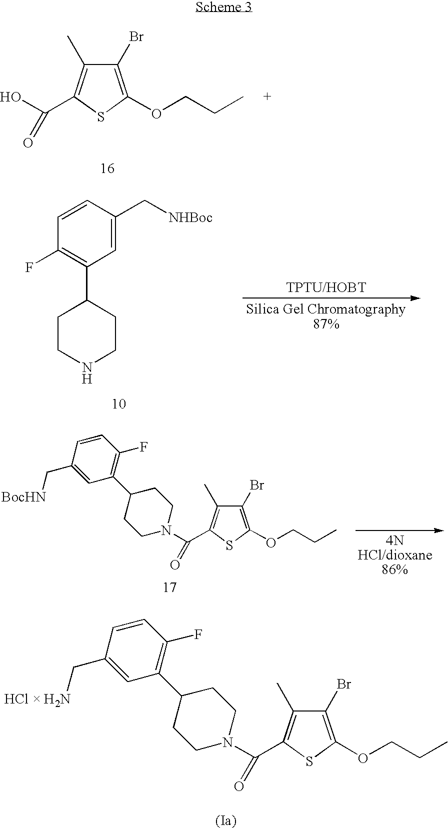

The compound of the present invention is basic, and such compound is useful in the form of the free base or in the form of a pharmaceutically acceptable acid addition salt thereof.

Acid addition salts may be a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Although pharmaceutically acceptable salts of said basic compound is preferred, all acid addition salts are useful as sources of the free base form even if the particular salt, per se, is desired only as an intermediate product as, for example, when the salt is formed only for purposes of purification, and identification, or when it is used as intermediate in preparing a pharmaceutically acceptable salt by ion exchange procedures. Pharmaceutically acceptable salts within the scope of the invention include those derived from mineral acids and organic acids, and include hydrohalides, e.g. hydrochloride and hydrobromide, sulfates, phosphates, nitrates, sulfamates, acetates, citrates, lactates, tartrates, malonates, oxalates, salicylates, propionates, succinates, fumarates, maleates, methylene-bis-b-hydroxynaphthoates, benzoates, tosylates, gentisates, isethionates, di-p-toluoyltartrates, methanesulfonates, ethanesulfonates, benzenesulfonates, p-toluenesulfonates, cyclohexylsulfamates and quinates. A more particular salt is salt of the compound of formula I is the hydrochloride salt.

As well as being useful in itself as an active compound, salts of the compound of the invention are useful for the purposes of purification of the compound, for example by exploitation of the solubility differences between the salts and the parent compound, side products and/or starting materials by techniques well known to those skilled in the art.

According to a further feature of the invention, the acid addition salt of the compound of this invention may be prepared by reaction of the free base with the appropriate acid, by the application or adaptation of known methods. For example, the acid addition salts of the compound of this invention may be prepared either by dissolving the free base in water or aqueous alcohol solution or other suitable solvents containing the appropriate acid and isolating the salt by evaporating the solution, or by reacting the free base and acid in an organic solvent, in which case the salt separates directly or can be obtained by concentration of the solution.

The acid addition salts of the compound of this invention can be regenerated from the salts by the application or adaptation of known methods. For example, the parent compound of the invention can be regenerated from their acid addition salts by treatment with an alkali, e.g. aqueous sodium bicarbonate solution or aqueous ammonia solution.

The starting materials and intermediates may be prepared by the application or adaptation of known methods, for example methods as described in the Reference Examples or their obvious chemical equivalents.

The present invention is also directed to some intermediates in the above schemes and, as such, the processes described herein for their preparation constitute further features of the present invention.

### EXAMPLES

The present invention may be better understood by reference to the following non-limiting Examples, which are provided as exemplary of the invention. The following examples are presented in order to more fully illustrate particular embodiments of the invention. They should in no way be construed, however, as limiting the broad scope of the invention.

In the nuclear magnetic resonance spectra (NMR), reported infra, the chemical shifts are expressed in ppm relative to tetramethylsilane. Abbreviations have the following significances: br=broad, dd=double doublet, s=singlet; m=multiplet.

### Example 1

### Step 1: Preparation of 1-(3-Bromo-4-fluoro-benzyl)-2,2,5,5-tetramethyl-[1,2,5]azadisilolidine (Compound 3)

110 g (0.46 mol) of 3-bromo-4-fluoro-benzylamine hydrochloride (2) is suspended in 900 mL of methylene chloride in a 2-L, three-neck, round-bottom flask equipped with N₂ blanket, Teflon-coated thermocouple temperature sensor, and mechanical stirring and cooled to ˜5° C. in an ice bath. A total of 144 g (1.42 mol, 3.1 equiv. d=0.7) is added resulting in a thick suspension. A solution of 1,2-bis(chlorodimethylsilyl) ethane (100 g, 0.46 mol) in 250 mL of methylene chloride is then added drop-wise over 1.5 hours while maintaining the temperature of the reaction mixture between 5 and 8° C. The mixture is stirred for 30 min and then warmed up to room temperature. The triethylamine hydrochloride suspension is filtered off. The filtrate is concentrated in vacuo (40° C., <50 mbar), 1 L of pentane is added and additional Et₃N×HCl precipitate that formed is filtered off. The procedure is repeated with another 1 L of pentane. The filtrate is concentrated in vacuo (40° C., <5 mbar) to a colorless liquid that solidifies upon cooling to give 159 g (˜100%) of compound 3 as a white crystalline solid.

Completion of the reaction should be monitored using 1H NMR to insure that any remaining starting material 2 and triethylamine hydrochloride is essentially absent as they consume n-butyl lithium in the subsequent step thereby resulting in a lower reaction yield.

### Step 2: Preparation of 1-Benzyl-4-[2-fluoro-5-(2,2,5,5-tetramethyl-[1,2,5]-azadisilolidin-1-ylmethyl)phenyl]piperidin-4-ol (Compound 5)

A solution of compound 3 (159 g, 0.4574 mol) in 1.5 L of anhydrous THF is placed in a 5-L, three-neck, round-bottom flask equipped with N₂ blanket, Teflon-coated thermocouple temperature sensor, and mechanical stirring and is cooled to −75° C. To this stirred solution is added drop-wise a 2.5 M solution of n-butyl lithium (192 ml, 0.48 mol, 1.05 equiv.) over about 1 hour while maintaining the reaction mixture temperature between −72° C. to −75° C. After 30 min, a solution of 1-benzyl-4-piperidone (Compound 4, 88.2 g, 0.47 mol, 1.02 equiv.) in anhydrous THF (350 mL+50 mL rinse) is added drop-wise over 1 h while maintaining the reaction temperature below −70° C. After stirring for 20 min at −70° C. to −75° C., 200 mL of methanol is added to the reaction mixture (color changes from orange to yellow), then the mixture is allowed to warm to room temperature. The reaction solution is concentrated in vacuo (50° C.) to give 295 g of brown oil, compound 5.

### Step 3: Preparation of 4-(5-Aminomethyl-2-fluorophenyl)-1-benzylpiperidin-4-ol (phosphate salt) (Compound 6)

The crude compound 5 (˜295 g) is diluted with 1 L of methylene chloride and transferred to a 3-L, three-neck, round-bottom flask equipped with Teflon-coated thermocouple temperature sensor, and mechanical stirring. To this solution is slowly added 53 g of 85% H₃PO₄ (1 equiv.) while stirring (exothermic) and the mixture is concentrated in vacuo (40° C.). 1 L of methylene chloride is added and the mixture is concentrated in vacuo (40° C., 1 mbar) to yellow foam, which is then slurried in 1 L of heptane. The solid is formed and isolated by filtration. The tan solid is dried to give 190 g of the compound 6. MS: m/z 315 (M+H) found.

### Step 4: Preparation of 3-(1-benzyl-1,2,3,6-tetrahydro-pyridin-4-yl)-4-fluoro-benzylamine (Compound 7)

A total of 190 g of the dried compound 6 is transferred to 2-L, three-neck, round-bottom flask equipped with Teflon-coated thermocouple temperature sensor, and mechanical stirring. A total of 600 mL of 85% H₃PO₄ is added. The resulting suspension is gradually heated to 100° C. while stirring. The reaction is monitored by HPLC for the reaction completion (typically 2 to 3 hours). Once the reaction completed, the reaction solution is cooled to room temperature, and diluted with 800 mL of water. The aqueous layer is washed with ether (2×200 mL). The aqueous layer is then neutralized with 50% aq. NaOH to pH>9 while maintaining temperature below 30° C. This aqueous solution is a very concentrated buffer system in which the pH remains unchanged at around 7-8 till the neutralization point is reached. Confirmation for complete neutralization is effected by adding a few drops of base to a small sample of the supernatant liquid to insure that no additional precipitation is observed. A large amount of salt precipitated out. The mixture is filtered and the solid is rinsed with DCM (methylene dichloride) (approximate 3 L) and 2 L of water. The organic layer (bottom) is separated and washed with water (2×1 L). The organic solution is concentrated in vacuo (40° C.) to give 107 g of brown oil, compound 7. MS: m/z 297 (M+H) found.

### Step 5: Preparation of [3-(1-Benzyl-1,2,3,6-tetrahydropyridin-4-yl)-4-fluoro-benzyl]carbamic acid tert-butyl ester (Compound 8)

To a 2-L, three-neck, round-bottom flask equipped with Teflon-coated thermocouple temperature sensor, and mechanical stirring is added 50 g of compound 7 and a solution of methanol (800 mL) and water (400 mL). 36.8 g of BOC anhydride and 2 mL of 50% NaOH are added and the mixture is stirred at room temperature. The product is precipitated out of the solution after 2 hours stirring. If the Boc product doesn't precipitate, decant the top aqueous layer and add n-heptane to the oil to solidify the product. The mixture is stirred overnight at room temperature. The solid is isolated by filtration and slurried in 1.05 L of MeOH/water (2:1 by volume) for 4 hours, then isolated by filtration and dried for 4 days to give 40 g (overall yield is about 35% to 48% from compound 3) of compound 8 as a light yellow solid. Purity: 96.3% by HPLC. MS: m/z 397 (M+H), 398 (M+2H).

### Step 6: Preparation of 6 (4-Fluoro-3-piperidin-4-yl-benzyl) carbamic acid tert-butyl ester, acetate salt (Compound 9)

64 g (0.16 mol) of compound 8, 6.4 g of palladium hydroxide/C 20%, 19.5 g (2 equiv.) of glacial acetic acid and 250 mL of methanol are charged to a 1-L hydrogenation vessel. The reaction mixture vessel is purged (N₂/vacuum, 3 times), then filled H₂ to 40 psi and shook overnight at room temperature. The catalyst is filtered off and the filtrate is concentrated in vacuo (40° C.) to yield an oil. 200 mL of isopropyl ether is added and stirred overnight. A white solid, which precipitates out, is filtered, rinsed with isopropyl ether and dried to give 53.5 g of white solid with 95.4% purity by HPLC. The solid is slurried in 500 mL of MTBE for 5 hours, then isolated by filtration, and slurried again in 500 mL of MTBE overnight. The solid is isolated by filtration and dried to afford 51.3 g (86.3%) white solid of the acetate salt of compound 9. Elemental analysis: Calculated for C17H25FN202: C, 61.94; H, 7.93; N, 7.6. Found: C, 62.0; H, 8.17; N, 7.49. KF: 0.34% water. HPLC: R₁ 9.14 min, purity 97% by AUC. MS: m/z 309 (M+H), 310 (M+2H).

### Step 7: Preparation of (4-Fluoro-3-piperidin-4-yl-benzyl) carbamic acid tert-butyl ester (Compound 10)

The acetate salt of compound 9 (51 g) is dissolved in 400 mL of water and the pH is adjusted to 5 with 2N HCl. The aqueous solution is washed with ether (2×200 mL). The aqueous layer is neutralized to pH>12 with 50% aq. NaOH. and extracted with ether (2×300 mL). The organic layer is washed with water and dried over Na2SO4, and then concentrated to oil. To the oil is added 200 mL of n-pentane and stirred for 3 hours. The product solid is isolated by filtration, washed with n-pentane, and dried at room temperature under house vacuum for 24 h to afford 42 g of compound 10 (98%) as a white solid. MS: (ESI) m/z 309 (M+H). Elemental analysis: Calculated for C17H25FN202: C, 66.21; H, 8.17; N, 9.08. Found without correction for water: C, 64.30; H, 8.64; N, 8.77. KF: 2.57% water. HPLC: R₁ 9.16 min, purity 98.1% by AUC.

Note: If the purity of the acetate salt compound 9 is less than 95% by HPLC, a solution of n-pentane and ether (up to 1:1 by volume) can be used to solidify the product (free base) from oil instead of only n-pentane. The product is very soluble in ether, so if more ether is used; then more product will be lost, thereby resulting in a lower yield.

### Example 2

### Step 1: Preparation of bis(propoxythiocarbonyl) sulfide (compound 11)

84.2 g power potassium hydroxide (1.27 mol) is added to 530 mL n-propanol placed in a three-necked round bottom flask equipped with a mechanical stirrer and a cooling bath at room temperature. 80 mL carbon disulfide (1.33 mol) is then added to the solution via a pressure-equalized additional funnel dropwise over 1 hour. The stirring is continued for 3 hours. 200 mL water is added. 73.5 g (0.6 mol) of propyl chloroformate is added in neat form dropwise via a pressure-equalized additional funnel. The mixture is stirred at room temperature overnight. The mixture is then diluted with 400 mL heptane. The aqueous layer is extracted with 100 mL heptane twice. The combined organic layer is washed with 100 mL water, 100 mL brine, then dried over potassium carbonate and evaporated. The residual propanol is further removed by high vacuum distillation to obtain a clear yellow liquid. 10 g of the clear yellow liquid is dissolved in 10 g of heptane and purified by silica gel chromatography, eluting with heptane to obtain 8.35 g of compound 11 (41% yield).

### Step 2: Preparation of 3-oxo-thiobutyric acid O-propyl ester (Compound 12)

1.82 g (45 mmol) sodium hydride is suspended in 40 mL toluene. To this suspension is added a solution of 1.74 g (30 mmol) acetone and 4.76 g (20 mmol) compound 11 in 10 mL toluene at 40° C. with stirring. The reaction is initiated by introducing small amount of potassium hydride. When doing so, bubbles evolve and the color of the reaction mixture changes from yellow to orange. The reaction mixture is stirred at 40° C. for one hour, and then cooled to 0° C. in an ice-water bath. The reaction mixture is then poured into a beaker containing 11 mL 4N HCl, ice and 150 mL ether. The organic layer is separated and concentrated. The crude product is purified by silica gel chromatography, eluting with 5% ethyl acetate in heptane to obtain 3-oxo-thiobutyric acid O-propyl ester (compound 12).

### Step 3: Preparation of (3-oxo-1-propoxy-but-1-enylsulfanyl)-acetic acid methyl ester (Compound 13)

Compound 12 is then dissolved in 40 mL DMF and cooled to 0° C. To this solution is added 4.6 g α-bromo methyl acetate in 5 mL DMF and 5.2 mL of triethylamine. A white precipitate is formed instantly. This suspension is stirred at 0° C. for 2 hours, and then is poured into ether/ice-water. The organic layer is separated and concentrated. The crude product is purified by silica gel chromatography, and then is eluted with 3% methanol in DCM to obtain 2.61 g of compound 13 (56% yield for two steps).

### Step 4: Preparation of 3-methyl-5-propoxy-thiophene-2-carboxylic acid methyl ester (compound 14)

A mixture of 2.61 g (11.24) compound 13 and 2 mL 0.5M sodium methoxide/methanol in 40 mL methanol is heated at 70-73° C. for 40 min. The mixture is then poured into ether/ice-water. The organic layer is separated and concentrated. The crude product is purified by silica gel chromatography, eluting with DCM to obtain 1.85 g (76.8 yield) of compound 14.

### Step 5: Preparation of 4-bromo-3-methyl-5-propoxy-thiophene-2-carboxylic acid methyl ester (compound 15)

To a solution of 1.22 g (5.7 mmol) compound 14 in 20 mL trichloromethane is added 11 mL 0.55M bromine/trichloromethane at 0° C., and stirred for 10 min. The mixture is quenched with sodium sulfite aqueous solution, and extracted with DCM. The organic layer is separated and concentrated. The crude product is purified by potassium carbonate-silica gel pad and rinsed with DCM to obtain 1.67 g (100% yield) of compound 15.

### Step 6: Preparation of 4-bromo-3-methyl-5-propoxy-thiophene-2-carboxylic acid (compound 16)

To a solution of 1.67 g of compound 15 (5.70 mmol) in 27 mL dioxane is added 10 mL of 2M LiOH/water and 9 mL water. The mixture is stirred at room temperature for 4 hours. The mixture is then diluted with 10 mL water and extracted with 10 mL ether twice. The aqueous solution is cooled in an ice-water bath and acidified with 4M HCl. The white solid is collected by suction filtration to provide 1.28 g (80.5% yield) of compound 16.

### Example 3

### Step 1: Preparation of {3-[1-(4-bromo-3-methyl-5-propoxy-thiophene-2-carbonyl)-piperidin-4-yl]-4-fluoro-benzyl}-carbamic acid tert-butyl ester (compound 17)

To a solution of 170 mg compound 16 (0.61 mmol) in 25 mL DCM under N₂ is added 170 mg TPTU (0.61 mmol) and 80 mg 1-hydroxy-1H-benzotriazole (HOBt) (0.61 mmol). The mixture is stirred for 3 minutes. To the mixture is then added a solution of 200 mg (0.65 mmol) of compound 10 in 5 mL DCM and 0.3 mL diisopropyl ethylamine (1.2 mmol). The mixture is stirred at room temperature for 24 hours. The mixture is then washed with 20 mL water, dried over anhydrous sodium sulfate and concentrated. The oil crude is purified by silica gel chromatography, eluting with 3% methanol in DCM to obtain 0.3 g (86.5% yield) of compound 17.

¹H NMR [CDCl₃]: δ (TMS) 7.14-7.04 (m, 2H), 6.95 (dd, H), 4.82 (br s, H), 4.38 (br d, 2H), 4.23 (d, 2H), 4.05 (t, 2H), 3.17-2.95 (m, 3H), 2.21 (s, 3H), 1.92-1.59 (m, 6H), 1.44 (s, 9H), 1.05 (t, 3H). MS(ESI+): 569 (M⁺+1).

### Step 2: Preparation of [4-(5-aminomethyl-2-fluoro-phenyl)-piperidin-1-yl]-(4-bromo-3-methyl-5-propoxy-thiophen-2-yl)-methanone hydrochloride (compound I)

A solution of 0.25 g compound 17 (0.44 mmol) in 8 mL 4M HCl/dioxane under nitrogen is stirred at room temperature for 3 hours. The solution is diluted with 30 mL ether and stirred for 5 min. The liquid is decanted from the solid. The solid is washed with 30 mL ether and the liquid is decanted again. The solid is then dissolved in 3% methanol in DCM and purified by silica gel chromatography, eluting with 3% to 10% methanol in DCM. Compound I, 0.19 g (86.4% yield), is obtained as an amorphous solid by removing the solvent from combined pure fractions of the product by evaporation. The product is an amorphous glass.

¹H NMR [CDCl₃]: δ(TMS) 8.62 (br s, 3H), 7.53-7.43 (m, H), 7.36-7.26 (m, H), 6.97 (dd, H), 4.22 (br d, 2H), 4.18-3.99 (m, 4H), 3.16-2.90 (m, 3H), 2.17 (s, 3H), 2.00-1.60 (m, 6H), 1.02 (t, 3H). MS(ESI+): 469 (M⁺+1). 100% purity by LC/MS (UV 220 nm and total ion count).

### BIOLOGICAL ACTIVITY

The properties of the compound of the present invention are demonstrated by: 1) its β-Tryptase Inhibitory Potency (IC₅₀ and Kᵢ values), and 2) its activity as measured in the Guinea Pig Model of Airway Hyperreactivity (Oral ED₅₀).

In Vitro Test Procedure

As all the actions of tryptase, as described in the background section, are dependent on its catalytic activity, then compounds that inhibit its catalytic activity will potentially inhibit the actions of tryptase. Inhibition of this catalytic activity may be measured by the in vitro enzyme assay and the cellular assay.

Tryptase inhibition activity is confirmed using either isolated human lung tryptase or recombinant human β tryptase expressed in yeast cells. Essentially equivalent results are obtained using isolated native enzyme or the expressed enzyme. The assay procedure employs a 96 well microplate (Costar 3590) using L-pyroglutamyl-L-prolyl-L-arginine-para-nitroanilide (S2366: Quadratech) as substrate (essentially as described by McEuen et. al. Biochem Pharm, 1996, 52, pages 331-340). Assays are performed at room temperature using 0.5mM substrate (2×Kₘ) and the microplate is read on a microplate reader (Beckman Biomek Plate reader) at 405 nm wavelength.

Materials and Methods for Tryptase Primary Screen (Chromogenic assay)

Assay buffer
50 mM Tris (pH 8.2), 100 mM NaCl, 0.05% Tween 20, 50 μg/mL heparin.

Substrate
S2366 (Stock solutions of 2.5 mM).

Enzyme
Purified recombinant beta Tryptase Stocks of 310 μg/mL.
   Protocol (Single Point Determination)
Add 60 μL of diluted substrate (final concentration of 500 μM in assay buffer) to each well
Add compound in duplicates, final concentration of 20 μM, volume 20 μL
Add enzyme at a final concentration of 50 ng/mL in a volume of 20 μL
Total volume for each well is 100 μL
Agitate briefly to mix and incubate at room temp in the dark for 30 minutes
Read absorbencies at 405 nM

Each plate has the following controls:
Totals: 60 μL of substrate, 20 μL of buffer (with 0.2% final concentration of DMSO), 20 μL of enzyme
Non-specific: 60 μL of substrate, 40 μL of buffer (with 0.2% DMSO)
Totals: 60 μL of substrate, 20 μL of buffer (No DMSO), 20 μL of enzyme
Non-specific: 60 μL of substrate, 40 μL of buffer (No DMSO)
   Protocol (IC₅₀ and Kᵢ determination)

The protocol is essentially the same as above except that the compound is added in duplicates at the following final concentrations: 0.01, 0.03, 0.1, 0.3, 1, 3, 10 μM (All dilutions carried out manually). For every assay, whether single point or IC₅₀ determination, a standard compound is used to derive IC₅₀ for comparison. From the IC₅₀ value, the Kᵢ can be calculated using the following formula: Kᵢ=IC₅₀/(1+[Substrate]/Kₘ).

The β-Tryptase inhibitory potency for the compound of formula I is IC₅₀ and Kᵢ values of 76 nM and 15 nM respectively.

In Vivo Test Procedure

Assay Protocol:

Sensitization and drug treatment: Male Hartley guinea pigs (225-250 g) are sensitized with ovalbumin (0.5 mL of 1% solution, i.p. and s.c.). On day 4, animals received a booster injection (i.p.) of 0.5 mL of 1% ovalbumin. On day 21, animals are orally dosed (2mL/kg) with either vehicle (0.5% methylcellulose/0.2% Tween 80) or test compound 2 hours prior to antigen challenge. Thirty minutes before antigen challenge the animals are also injected with mepyramine (30 mg/kg, i.p.) to prevent anaphylactic collapse. Animals are then exposed for 5 minutes to an aerosol of either saline (control animals) or 1% ovalbumin using a deVilbiss Ultraneb nebulizer.

AHR measurement: Eighteen to twenty four hours after challenge, animals are anesthetized with a combination of ketamine (133 mg/kg) and xylazine (24 mg/kg) given intramuscularly, surgically prepared and then mounted in a whole body plethysmograph for lung function measurement. Animals are connected to Ugo-Basile ventilators delivering a tidal volume of 1 mL/100 g at a rate of 50 breaths/minute via a tracheal cannula. The jugular vein is also cannulated for histamine challenge. A water filled esophageal cannula is placed such that transpulmonary pressure is recordable. Transpulmonary pressure is measured as the difference between the tracheal and esophageal cannulas using a differential pressure transducer. The volume, airflow, and transpulmonary pressure signals are monitored using a pulmonary analysis system (Buxco XA software) and used to calculate pulmonary resistance (cm H₂O/mL/s) and dynamic compliance (mL/cm H₂O). Airway resistance and dynamic compliance are computed on a breath by breath basis. Histamine is administered intravenously and reactivity to increasing concentrations (0.3-20 μg/kg) assessed. ED₅₀'s are estimated from the area under the curve (AUC) values derived from the individual histamine dose-response curves.

Plasma and Lung Drug Levels Plasma and lung compound levels are determined in satellite groups. Three to four guinea pigs from each of the experimental-drug treatment groups are used for the determination of drug levels. At the indicated time point, (either 2- or 24 hours after dosing), animals are euthanized, and 1 mL blood samples are obtained by cardiac puncture and collected into heparin-coated syringes containing 20 μL (per 1 mL of blood) of a 5 mM hydralazine solution. Plasma is separated from the cellular component of the blood by centrifugation, and stored at −20° C. until assayed. Lung samples are dissected free of connective tissues, blotted dry, weighed and stored in 20 mL vials containing 5 mL of a 5 mM hydralazine solution in saline. The frozen plasma samples are then transferred on dry ice to the Pilot PK group for compound levels determination.

Results:

Efficacy of compound I is profiled on Airway Hyperresponsiveness (AHR) to histamine in sensitized guinea pigs, via oral route. Compound I has no effect on basal airway resistance or basal dynamic lung compliance.

Sensitization and single challenge with ovalbumin results in an increase in bronchial reactivity to histamine as denoted by a leftward shift in the dose-response curve of the spasmogen and also by a significant increase in the area under the curve (AUC) for both airway resistance and lung compliance. Absolute values represent an increase in airway resistance and a decrease in lung compliance.

Upon oral dosing, 2 hours prior to ovalbumin challenge, compound I significantly protects against antigen-stimulated AHR to histamine with an ED₅₀=0.1 mg/kg as measured by airway resistance and dynamic lung compliance.

In separate animals, compound I levels are measured in bronchoalveaolar lavage fluid (BAL), lung and plasma, 2 hours after compound dosing (1 mg/kg, p.o.). There is an appreciable amount of compound I in target organs such lung and BAL (not taking into account dilution factor for BAL). Plasma compound levels are detected at a much lower level.

In satellite animals, compound I levels, in both lung and plasma, are also measured 24 hours after dosing. While no plasma could be detected, there is, however, a dose-dependent appreciable amount of compound I detected in guinea pig lung 24 hours after dosing. Compound I is also shown to possess a long duration of action with an average ED₅₀ of 0.4 mg/kg when dosed 24 hours prior to antigen challenge. This long duration of action is in agreement with its extended long exposure.

The oral data of the compound of the present invention in the guinea pig model of airway hyperresponsiveness clearly shows that the compound exhibits tryptase inhibition activity. Consequently, the compound of the present invention readily has application as a pharmaceutical for treating a wide variety of tryptase related conditions, and naturally, in methods for treating such conditions in a patient.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

Various publications are cited herein, the disclosures of which are incorporated by reference in their entireties.

What is claimed is:

1. A compound of formula I: 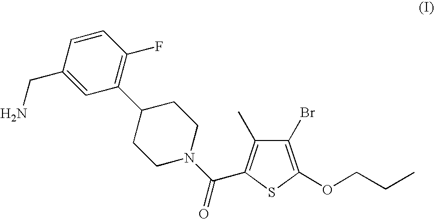
or a prodrug, pharmaceutically acceptable salt, or solvate thereof.

2. The compound of claim 1 as a pharmaceutically acceptable salt thereof.

3. The compound of claim 2 wherein the pharmaceutically acceptable salt is a hydrochloride.

4. A method for treating a patient suffering from, or subject to, a physiological condition in need of amelioration of an inhibitor of tryptase comprising administering to the patient a therapeutically effective amount of the compound of claim 1.

5. The method of claim 4, wherein the physiological condition is selected from the group consisting of inflammatory disease, a disease of joint cartilage destruction, ocular conjunctivitis, vernal conjunctivitis, inflammatory bowel disease, asthma, allergic rhinitis, interstitial lung disease, fibrosis, sceleroderma, pulmonary fibrosis, liver cirrhosis, myocardial fibrosis, neurofibroma, hypertrophic scar, dermatological condition, condition related to atherosclerotic plaque rupture, periodontal disease, diabetic retinopathy, tumor growth, anaphylaxis, multiple sclerosis, peptic ulcer, and syncytial viral infection.

6. The method of claim 5, wherein the physiological condition is inflammatory disease.

7. The method of claim 6 wherein the inflammatory disease is joint inflammation, arthritis, rheumatoid arthritis, rheumatoid spondylitis, gouty arthritis, traumatic arthritis, rubella arthritis, psoriatic arthritis, or osteoarthritis.

8. The method of claim 5, wherein the physiological condition is COPD.

9. The method of claim 5, wherein the physiological condition is COPD exacerbations.

10. The method of claim 5, wherein the physiological condition is a dermatological condition.

11. The method of claim 10, wherein the dermatological condition is atopic dermatitis or psoriasis.

12. The method of claim 5, wherein the physiological condition is related to atherosclerotic plaque rupture.

13. The method of claim 12, wherein the atherosclerotic plaque rupture is consequent to myocardial infarction, stroke, or angina.

14. A method for treating a patient suffering from asthma, comprising administering to the patient a combination of a therapeutically effective amount of a compound of claim 1, and a second compound selected from the group consisting of a beta andrenergic agonist, anticholinergic, anti-inflammatory corticosteroid, and anti-inflammatory agent.

15. The method of claim 4, wherein the administering is such that the compound of claim 1 is preferentially distributed to lung tissue versus plasma.

16. A pharmaceutical composition comprising a therapeutically effective amount of a compound of claim 1 and a pharmaceutically acceptable carrier thereof.

17. A pharmaceutical composition comprising a compound of claim 1 and a therapeutically effective amount of a second compound selected from the group consisting of a beta andrenergic agonist, anticholinergic, anti-inflammatory corticosteroid, and anti-inflammatory agent; and a pharmaceutically acceptable carrier.

18. The pharmaceutical composition of claim 17, wherein the second compound is a beta andrenergic agonist.

19. The pharmaceutical composition of claim 18, wherein the beta andrenergic agonist is selected from albuterol, terbutaline, formoterol, fenoterol, or prenaline.

20. The pharmaceutical composition of claim 17, wherein the second compound is an anticholinergic.

21. The pharmaceutical composition of claim 20, wherein the anticholinergic is ipratropium bromide.

22. The pharmaceutical composition of claim 17, wherein the second compound is an anti-inflammatory corticosteroid.

23. The pharmaceutical composition of claim 22, wherein the anti-inflammatory corticosteroid is selected from beclomethasone dipropionate, triamcinolone acetonide, flunisolide or dexamethasone.

24. The pharmaceutical composition of claim 17, wherein the second compound is an anti-inflammatory agent.

25. The pharmaceutical composition of claim 24, wherein the anti-inflammatory agent is sodium cromoglycate or nedocromil sodium.

26. The pharmaceutical composition of claim 17, wherein the second compound is a pharmaceutically acceptable carrier thereof.
